(19) United States
(12) Patent Application Publication
(10) Pub. No.: US 2009/0264848 A1
(43) Pub. Date: Oct. 22, 2009

(54) Methodology for presenting competitor favoritism

(76) Inventors: John J. D'Auria, Bernardsville, NJ (US); Nell T. D'Auria, Bernardsville, NJ (US)

Correspondence Address: John J. D'Auria, 79 Ambar Place, Bernardsville, NJ, 07924, US

(21) Appl. No.: 12/148,647

(22) Filed: Apr. 21, 2008

(51) Int. Cl. A61F 13/15

(52) U.S. Cl. 604/385.01

(57) ABSTRACT

The present invention is a methodology for presenting competitor favoritism, which compromises: (a.) presenting a diaper having a front and a rear, the diaper graphically illustrating number one in any known language, numeral system or symbol, on the front of the diaper and number two on the rear of the diaper; (b.) displaying a favored competitor from two or more competitors on the front of the diaper in proximity to the number one graphic illustration; and, (c.) displaying at least one disfavored competitor from the two or more competitors on the rear of the diaper in proximity to the number two. Furthermore, the present invention may be utilized for bottom wear, including underwear, shorts and pants.

### BACKGROUND OF INVENTION

a. Field of Invention

The invention relates generally to diapers and bottom wear which includes, bottoms underwear, shorts and pants. More specifically, there is a graphical illustration of number one and number two, which are associated with a display of a favored competitor and a disfavored competitor, respectively. The number one and favored competitor is located on the front of the diapers and bottom wear, while the number two and disfavored competitor is locator on the rear of the diapers and bottom wear.

b. Description of Related Art

The following patents are representative of diaper and related patents:

U.S. Pat. No. 7,332,642 issued to Kuang-Kai Liu on Feb. 19, 2008 describes an invention that relates to a disposable absorbent article comprising a wetness indicator disposed between the absorbent core and the backsheet and in liquid communication with the absorbent core; the wetness indicator comprising a central graphic and a background graphic; wherein the background graphic comprises at least one responsive color composition that, upon wetting, exhibits a visible change that is selected from the group consisting of a color change, a graphic change and combinations thereof.

U.S. Pat. No. 7,313,929 issued to Susan Lazor on Jan. 1, 2008 describes a user wearable device for tracking or reminding the user of time and/or numerical records. In the preferred embodiments, the device takes the form of an attractive bracelet. The bracelet is made of two sets of beads strung together to form the bracelet and at least one charm for removable placement between the beads. The first set of beads has numbers marked thereon while the second set of beads does not have any markings. The two sets of beads are placed in a predetermined alternate fashion along the bracelet such that the at least one charm can be placed between certain beads to note the time and/or numerical record. In the preferred embodiment each of the twelve sequentially numerically marked beads is separated by a predetermined number of unmarked beads, such as 4, such that each unmarked beads represents a fifteen (15) minute interval between the hours.

U.S. Pat. No. 6,250,929 issued to Thomas Mathias Kolb et al. on Jun. 26, 2001 describes a method for improving the effectiveness of a child's toilet training regime utilizes a progress scale to evaluate the child's current level or stage of toilet training. Recommendations for improving the effectiveness of the child's training regime are divided into a plurality of unique groups corresponding to a plurality of stages of toilet training. The child's toilet training progress value, determined using the progress scale, is matched to a particular stage of training and the unique group of recommendations corresponding to the child's stage of training are selected. Methods for comparing the effectiveness of various toilet training regimes are also disclosed.

U.S. Pat. No. 5,690,624 issued to Toru Sasaki et al. on Nov. 25, 1997 describes a disposable diaper that is provided with an indicator adapted to be revealed as it is wetted with discharged urine and to tell a mother her baby's urination. The indicator comprises a hydrophilic base sheet, a first coating layer formed on one side of the base sheet so as to develop its proper color as the indicator is wetted with urine, and a second coating layer formed on the other side of the base sheet or upper surface of the first coating layer and intermittently bonded to the inner surface of the backsheet so that the second coating layer covers up the first coating layer. The second coating layer becomes transparent as the indicator is wetted with urine.

U.S. Pat. No. 5,171,308 issued to Francis G. Gallagher et al. on Dec. 15, 1992 describes an invention which provides novel polyesters, fibers and films, nonwovens from the fibers and disposable products of the polyesters such as diapers. The products are degradable under the conditions typically existing in waste composting processes, have low ingredient costs and yet provide strength and toughness properties adequate for end uses such as in disposable diapers. The polyesters are based upon polyethylene terphthalate copolymerized with a non-aromatic diacid, such as adipic and glutaric acids, and containing alkali metal or alkaline earth metal sulfo groups, such as a metal 5-sulfoisophthalic acid derivative.

U.S. Pat. No. 4,249,532 issued to Herbert Polansky, et al. on Feb. 10, 1981 describes a disposable diaper that shows a decorative printed design on its one side, the design being printed in reverse and on the inner side of a transparent polyethylene sheet that forms the one side of the diaper.

Notwithstanding the prior art, the present invention is neither taught nor rendered obvious thereby.

### SUMMARY OF INVENTION

The present invention is a methodology for presenting competitor favoritism, which compromises: (a.) presenting a diaper having a front and a rear, the diaper graphically illustrating number one in any known language, numeral system or symbol, on the front of the diaper and number two on the rear of the diaper; (b.) displaying a favored competitor from two or more competitors on the front of the diaper in proximity to the number one graphic illustration; and, (c.) displaying at least one disfavored competitor from the two or more competitors on the rear of the diaper in proximity to the number two.

In some embodiments of the present invention methodology for presenting competitor favoritism, the favored competitor is displayed to include a person's name.

In some embodiments of the present invention methodology for presenting competitor favoritism, the favored competitor is displayed to include a name representing an entity selected from the group consisting of a geographic location, a business entity, an institution, and a service organization.

In some embodiments of the present invention methodology for presenting competitor favoritism, the favored competitor is a sports team.

In some embodiments of the present invention methodology for presenting competitor favoritism, the at least one disfavored competitor is displayed as a generic catchall.

In some embodiments of the present invention methodology for presenting competitor favoritism, the generic catchall is selected from the group consisting of “everyone else”, “our competitors”, “the other guy”, “the other team” and “the rest of the world”. Others, such as “you” or “you know who” could be used.

In some embodiments of the present invention methodology for presenting competitor favoritism, the number one is graphically illustrated in a format selected from the group consisting of “NUMBER ONE”, “NUMBER 1”, “No. 1”, “# 1” and “No. One”. Alternatively, symbols such as one finger and two fingers could be used for number one and number two.

In some embodiments of the present invention methodology for presenting competitor favoritism, the graphically illustrated presentations are printed graphically illustrated presentations and the displays are printed displays. Heat transfer formats, imprints, silk screens, stamping and drawing are to be included among printing techniques.

In some embodiments of the present invention methodology for presenting competitor favoritism, the graphically illustrated presentations are sewn graphically illustrated presentations and the displays are sewn displays, including embroideries.

In some embodiments of the present invention methodology for presenting competitor favoritism, the graphically illustrated presentations are patch graphically illustrated presentations and the displays are patch displays.

In some other preferred embodiments of the present invention methodology for presenting competitor favoritism, the methodology for presenting competitor favoritism, which compromises: (a.) presenting bottom wear selected from the group consisting of underwear, shorts and pants having a front and a rear, the bottom wear graphically illustrating number one in any known language, numeral system or symbol, on the front of the bottom wear and number two on the rear of the bottom wear; (b.) displaying below the waist area, a favored competitor from two or more competitors on the front of the bottom wear in proximity to the number one graphic illustration; and, (c.) displaying below the waist area at least one disfavored competitor from the two or more competitors on the rear of the bottom wear in proximity to the number two. Pants shall include conventional pants, such as two-legged, waist high outerwear, as well as any clothing item that includes a part or component containing legs and private part coverage, i.e. jumpsuits.

In some embodiments of the present invention methodology for presenting competitor favoritism of paragraph [00020], the favored competitor is displayed to include a person's name.

In some embodiments of the present invention methodology for presenting competitor favoritism of paragraph [00020], the favored competitor is displayed to include a name representing an entity selected from the group consisting of a geographic location, a business entity, an institution, and a service organization.

In some embodiments of the present invention methodology for presenting competitor favoritism of paragraph [00020], the favored competitor is a sports team.

In some embodiments of the present invention methodology for presenting competitor favoritism of paragraph [00020], the at least one disfavored competitor is displayed as a generic catchall.

In some embodiments of the present invention methodology for presenting competitor favoritism of paragraph [00020], the generic catchall is selected from the group consisting of “everyone else”, “our competitors”, “the other guy”, “the other team” and “the rest of the world”. Others, such as “you” or “you know who” could be used.

In some embodiments of the present invention methodology for presenting competitor favoritism of paragraph [00020], the number one is graphically illustrated in a format selected from the group consisting of “NUMBER ONE”, “NUMBER 1”, “No. 1”, “# 1” and “No. One”. Alternatively, symbols such as one finger and two fingers could be used for number one and number two.

In some embodiments of the present invention methodology for presenting competitor favoritism of paragraph [00020], the graphically illustrated presentations are printed graphically illustrated presentations and the displays are printed displays. Heat transfer formats, imprints, silk screens, stamping and drawing are to be included among printing techniques.

In some embodiments of the present invention methodology for presenting competitor favoritism of paragraph [00020], the graphically illustrated presentations are sewn graphically illustrated presentations and the displays are sewn displays, including embroideries.

In some embodiments of the present invention methodology for presenting competitor favoritism of paragraph [00020], the graphically illustrated presentations are patch graphically illustrated presentations and the displays are patch displays.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 is a top view of an embodiment of a present invention methodology for presenting competitors, illustrating a diaper;

FIG. 2 is a top view of another embodiment of a present invention methodology for presenting competitors, illustrating a diaper;

FIG. 3 is a front view of another embodiment of a present invention methodology for presenting competitors, illustrating bottoms underwear;

FIG. 4 is a rear view of the embodiment shown in FIG. 3;

FIG. 5 is a front view of another embodiment of a present invention methodology for presenting competitors, illustrating shorts;

FIG. 6 is a rear view of the embodiment shown in FIG. 5;

FIG. 7 is a front view of another embodiment of a present invention methodology for presenting competitors, illustrating pants;

FIG. 8 is a rear view of the embodiment shown in FIG. 7; and

FIG. 9 is a generic list of categories for graphical illustrations of a present invention methodology for presenting competitors.

### DETAILED DESCRIPTION OF THE EMBODIMENTS

The present invention relates to diapers and bottom wear which includes, bottoms underwear, shorts and pants. There is a graphical illustration of number one and number two, which are associated with a display of a favored competitor and a disfavored competitor, respectively. The number one and favored competitor is located on the front of the diapers and bottom wear, while the number two and disfavored competitor is locator on the rear of the diapers and bottom wear.

The present invention methodology for presenting competitor favoritism, which includes: (a.) presenting a bottom wear having a front and a rear, the bottom wear graphically illustrating number one in any known language, numeral system or symbol, on the front of the bottom wear and number two on the rear of the bottom wear; (b.) displaying a favored competitor from two or more competitors on the front of the bottom wear in proximity to the number one graphic illustration; and, (c.) displaying at least one disfavored competitor from the two or more competitors on the rear of the bottom wear in proximity to the number two.

The favored competitor is displayed may include a person's name or a sports team. Moreover, the favored competitor is displayed to include a name representing an entity selected from the group consisting of a geographic location, a business entity, an institution, and a service organization.

The at least one disfavored competitor may be displayed as a generic catchall which may be selected from the group consisting of “everyone else”, “our competitors”, “the other guy”, “the other team” and “the rest of the world”.

The number one may be graphically illustrated in a format selected from the group consisting of “NUMBER ONE”, “NUMBER 1”, “No. 1”, “# 1” and “No. One”.

In some embodiments of the present invention methodology for presenting competitor favoritism, the graphically illustrated presentations may be printed graphically illustrated presentations and the displays may be printed displays. Furthermore, the graphically illustrated presentations may be sewn graphically illustrated presentations or patch graphically illustrated presentations. The displays may be sewn displays or patch displays.

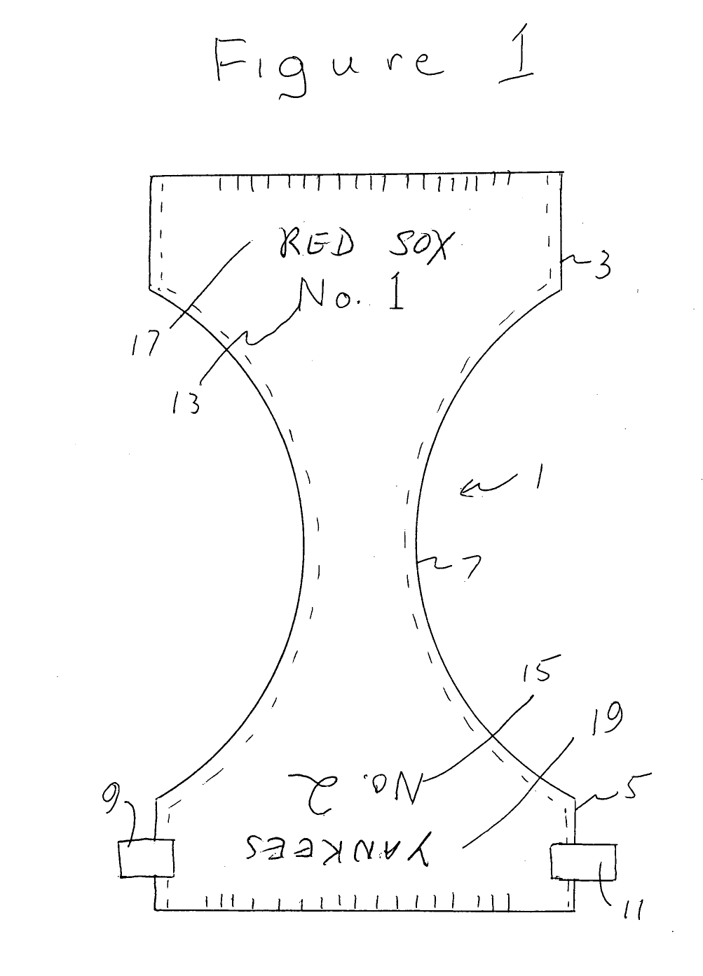

Referring now to the drawings wherein like reference numerals designate corresponding parts throughout the several views, FIG. 1 is a top view of an embodiment of a present invention methodology for presenting competitors 1, illustrating a diaper 7. The diaper 7 may be a disposable diaper, a cotton or other fiber reusable diaper, a synthetic reusable diaper or diaper made of a blend of materials.

The methodology for presenting competitor favoritism 1 includes presenting the diaper 7 having a front 3 and a rear 5. The rear 5 includes tabs 9, 11 for attachment to the front 3 of the diaper 7. The diaper 7 graphically illustrates number one in any known language, numeral system or symbol, on the front 3 of the diaper 7 and number two on the rear 5 of the diaper 7. As shown, number 1 is “No. 1” 13 and number two is “No. 2” 15. The number one is graphically illustrated in a format selected from the group consisting of “NUMBER ONE”, “NUMBER 1”, “No. 1”, “# 1” and “No. One”. The number two is graphically illustrated in a format selected from the group consisting of “NUMBER TWO”, “NUMBER 2”, “No. 2”, “# 2”, “NUMERO DUO” and “No. Two”. The graphically illustrated presentation 13, 15 is sewn, but also may be a patch display or printed.

Next, the methodology includes displaying a favored competitor 17 from two or more competitors on the front 3 of the diaper 7 in proximity to the number one graphic illustration 13. As shown, the favored competitor 17 is RED SOX, which is a sports baseball team. The favored competitor may include a name representing an entity selected from the group consisting of a geographic location, a business entity, an institution, and a service organization. The display 17 is a sewn display, but also may be a patch display or printed.

Finally, the methodology includes displaying at least one disfavored competitor 19 from the two or more competitors on the rear 5 of the diaper 7 in proximity to the number two 15. As shown the disfavored competitor 19 is YANKEES. The at least one disfavored competitor may be displayed as a generic catchall that is selected from the group consisting of “everyone else”, “our competitors”, “the other guy”, “the other team” and “the rest of the world”.

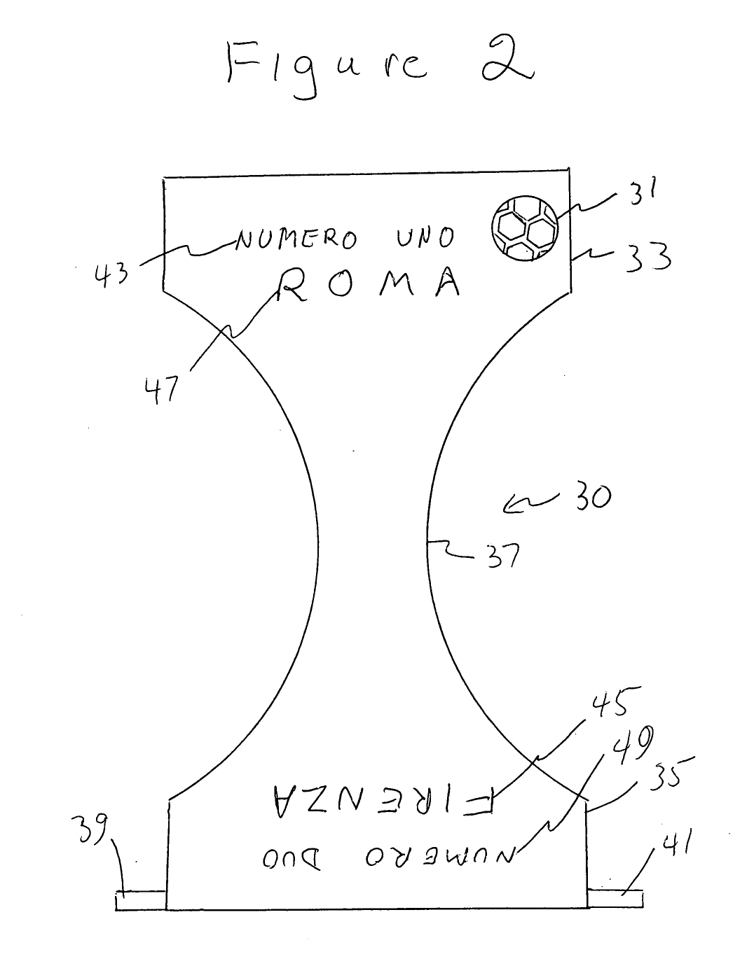

FIG. 2 is a top view of another embodiment of a present invention methodology for presenting competitors 30, illustrating a diaper 37. The methodology for presenting competitor favoritism 30 includes presenting the diaper 37 having a front 33 and a rear 35. The rear 35 includes tabs 39, 41 for attachment to the front 33 of the diaper 37. The diaper 37 graphically illustrates number one in any known language, numeral system or symbol, on the front 33 of the diaper 37 and number two on the rear 35 of the diaper 37. As shown, number 1 is “NUMERO UNO” 43 and number two is “NUMERO DUO” 49. The number one is graphically illustrated in a format selected from the group consisting of “NUMBER ONE”, “NUMBER 1”, “No. 1”, “# 1” and “No. One”. The number two is graphically illustrated in a format selected from the group consisting of “NUMBER TWO”, “NUMBER 2”, “No. 2”, “# 2”, “NUMERO DUO” and “No. Two”. The graphically illustrated presentation 43 is printed, but also may be a patch display or sewn. There is also illustrated a patch display 31 of a soccer ball on the front 33 of the diaper 37.

Next, the methodology includes displaying a favored competitor 47 from two or more competitors on the front 33 of the diaper 47 in proximity to the number one graphic illustration 13. As shown, the favored competitor 47 is a soccer team city, ROMA. The favored competitor may include a name representing an entity selected from the group consisting of a geographic location, a business entity, an institution, and a service organization. The display 47 is a printed display, but also may be a patch display or a sewn display.

Finally, the methodology includes displaying at least one disfavored competitor 45 from the two or more competitors on the rear 35 of the diaper 37 in proximity to the number two 49. As shown the disfavored competitor 45 is a soccer team city, FIRENZA. The at least one disfavored competitor 45 may be displayed as a generic catchall that is selected from the group consisting of “everyone else”, “our competitors”, “the other guy”, “the other team” and “the rest of the world”.

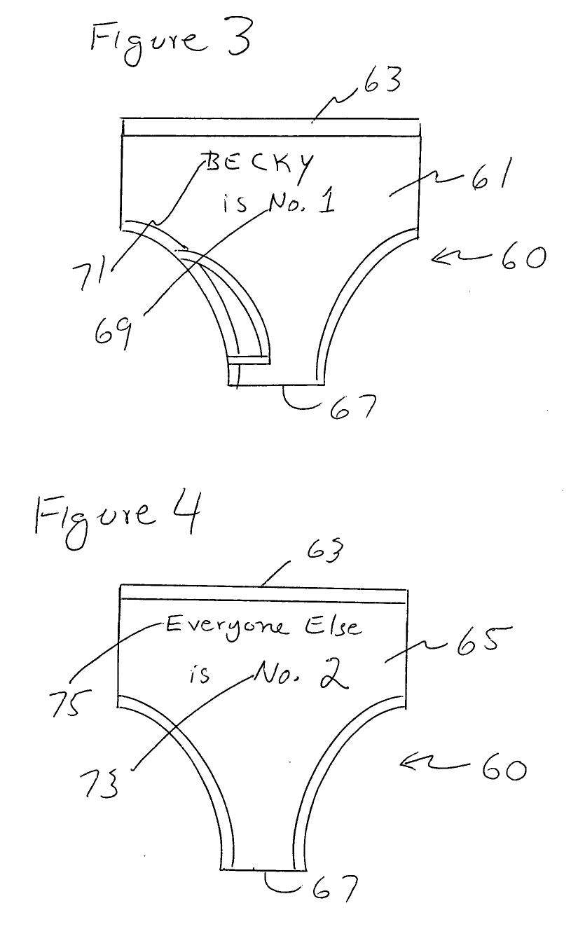

FIG. 3 is a front view of another embodiment of a present invention methodology for presenting competitors 60, illustrating bottoms underwear, while FIG. 4 is a rear view thereof.

The methodology for presenting competitor favoritism 60 includes presenting the bottoms underwear 67 having a front 61 and a rear 65. There is an elastic waist band 63 at the top for holding the bottoms underwear 67 in place when the bottoms underwear is placed upon a wearer. The bottoms underwear are sized and shaped to fit women, men and children. The bottoms underwear 67 graphically illustrates number one 69 in any known language, numeral system or symbol, on the front 61 of the bottoms underwear 67 and number two 73 on the rear 65 of the bottoms underwear 67. As shown, number 1 is “No. 1” 69 and number two is “No. 2” 73. The number one is graphically illustrated in a format selected from the group consisting of “NUMBER ONE”, “NUMBER 1”, “No. 1”, “# 1”, “NUMERO UNO” and “No. One”. The number two is graphically illustrated in a format selected from the group consisting of “NUMBER TWO”, “NUMBER 2”, “No. 2”, “# 2”, “NUMERO DUO” and “No. Two”. The graphically illustrated presentation 69 is printed, but also may be a patch display or sewn.

Next, the methodology includes displaying a favored competitor 71 from two or more competitors on the front 61 of the bottoms underwear 67 in proximity to the number one graphic illustration 69. As shown, the favored competitor 71 is a proper name, BECKY. The favored competitor 71 may include a name representing an entity selected from the group consisting of a geographic location, a business entity, an institution, and a service organization. The display 71 is a printed display, but also may be a patch display or sewn.

Finally, the methodology includes displaying at least one disfavored competitor 75 from the two or more competitors on the rear 65 of the bottoms underwear 67 in proximity to the number two 73. As shown, the disfavored competitor 75 is a generic catchall, “Everyone Else”. The at least one disfavored competitor 75 may be selected from the group consisting of “everyone else”, “our competitors”, “the other guy”, “the other team” and “the rest of the world”.

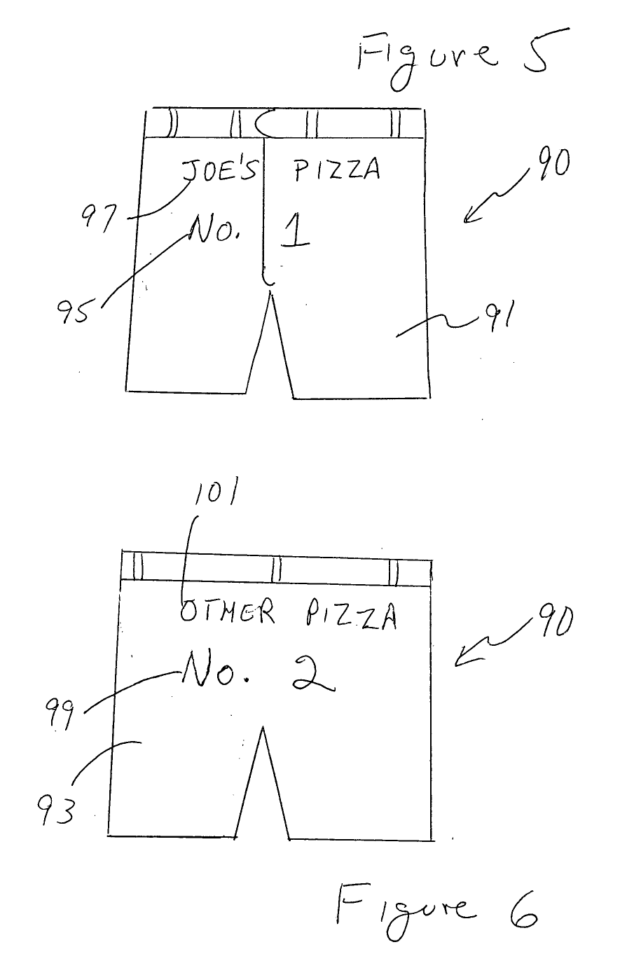

FIG. 5 is a front view of another embodiment of a present invention methodology for presenting competitors, illustrating shorts 90, while FIG. 6 is a rear view thereof.

The methodology for presenting competitor favoritism includes presenting the shorts having a front 91 and a rear 93. The shorts 90 are sized and shaped to fit women, men and children. The shorts 90 graphically illustrates number one 95 in any known language, numeral system or symbol, on the front 91 of the shorts 90 and number two 99 on the rear 93 of the shorts 90. As shown, number 1 is “No. 1” 95 and number two is “No. 2” 99. The number one is graphically illustrated in a format selected from the group consisting of “NUMBER ONE”, “NUMBER 1”, “No. 1”, “# 1”, “NUMERO UNO” and “No. One”. The number two is graphically illustrated in a format selected from the group consisting of “NUMBER TWO”, “NUMBER 2”, “No. 2”, “# 2”, “NUMERO DUO” and “No. Two”. The graphically illustrated presentation 93, 95 is printed, but also may be a patch display or sewn.

Next, the methodology includes displaying a favored competitor 97 from two or more competitors on the front 91 of the shorts 90 in proximity to the number one graphic illustration 95. As shown, the favored competitor 97 is a business, JOE'S PIZZA. The favored competitor 97 may include a name representing an entity selected from the group consisting of a geographic location, a business entity, an institution, and a service organization. The display 97 is a printed display, but also may be a patch display or sewn.

Finally, the methodology includes displaying at least one disfavored competitor 101 from the two or more competitors on the rear 93 of the shorts 90 in proximity to the number two 99. As shown, the disfavored competitor 101 is a generic catchall, “OTHER PIZZA”. The at least one disfavored competitor 101 may be selected from the group consisting of “everyone else”, “our competitors”, “the other guy”, “the other team” and “the rest of the world”.

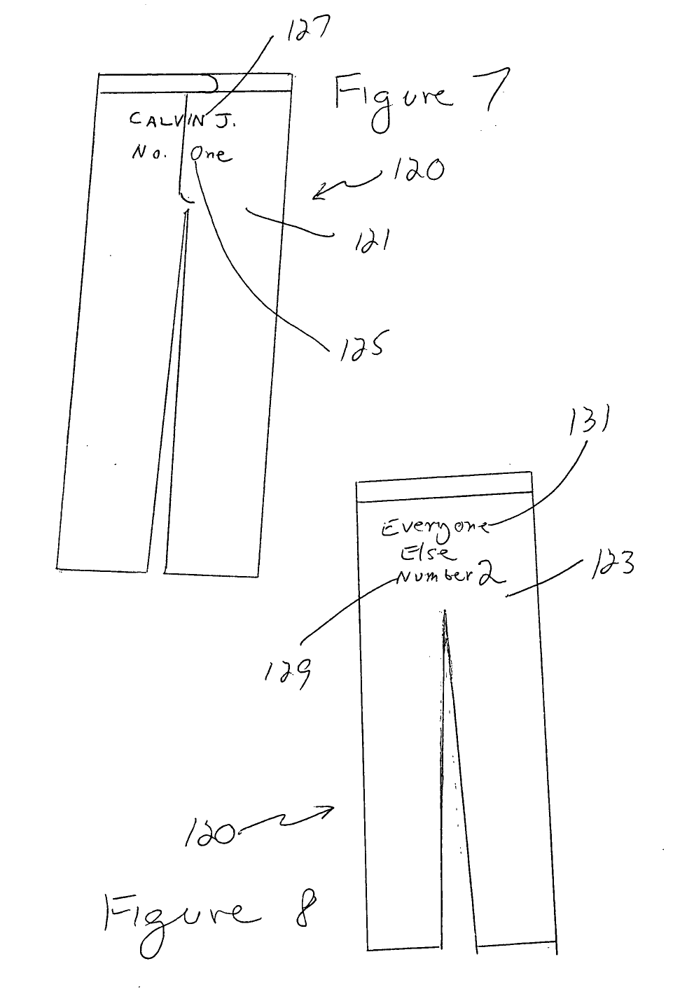

FIG. 7 is a front view of another embodiment of a present invention methodology for presenting competitors, illustrating pants 120, while FIG. 8 is a rear view thereof.

The methodology for presenting competitor favoritism includes presenting the pants having a front 121 and a rear 123. The pants 120 are sized and shaped to fit women, men and children. The pants 120 graphically illustrates number one 125 in any known language, numeral system or symbol, on the front 121 of the pants 120 and number two 129 on the rear 123 of the pants 120. As shown, number 1 is “No. One” 125 and number two is “Number 2” 129. The number one is graphically illustrated in a format selected from the group consisting of “NUMBER ONE”, “NUMBER 1”, “No. 1”, “# 1”, “NUMERO UNO” and “No. One”. The number two is graphically illustrated in a format selected from the group consisting of “NUMBER TWO”, “NUMBER 2”, “No. 2”, “# 2”, “NUMERO DUO” and “No. Two”. The graphically illustrated presentation 125, 129 is printed, but also may be a patch display or sewn.

Next, the methodology includes displaying a favored competitor 127 from two or more competitors on the front 121 of the pants 120 in proximity to the number one graphic illustration 125. As shown, the favored competitor 127 is a proper name, CALVIN J. The favored competitor 127 may include a name representing an entity selected from the group consisting of a geographic location, a business entity, an institution, and a service organization. The display 127 is a printed display, but also may be a patch display or sewn.

Finally, the methodology includes displaying at least one disfavored competitor 131 from the two or more competitors on the rear 123 of the pants 120 in proximity to the number two 129. As shown, the disfavored competitor 131 is a generic catchall, “Everyone Else”. The at least one disfavored competitor 131 may be selected from the group consisting of “everyone else”, “our competitors”, “the other guy”, “the other team” and “the rest of the world”.

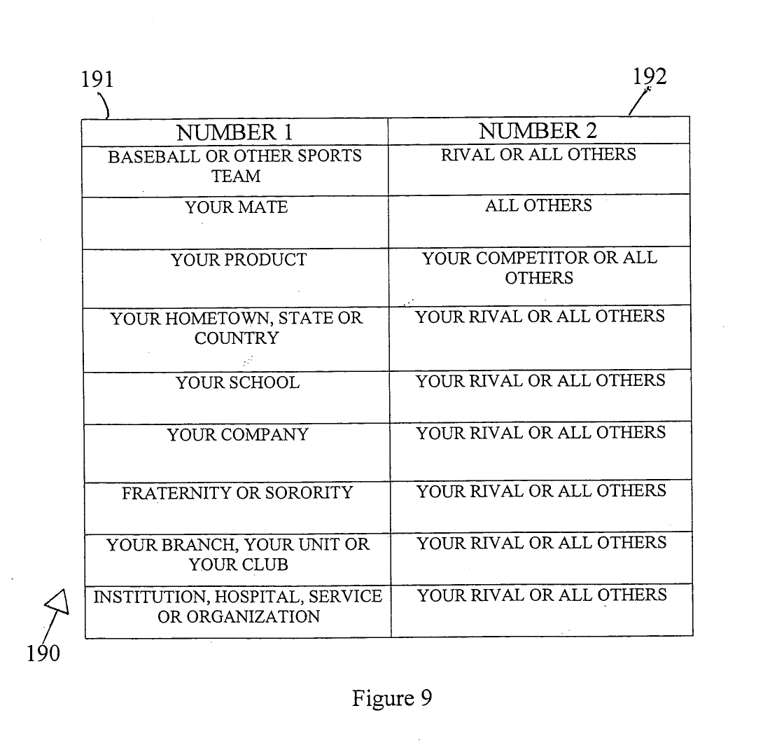

FIG. 9 is a generic list of categories 190 for graphical illustrations of a present invention methodology for presenting competitors for displays of “NUMBER 1” 191 and “NUMBER 2” 192.

NUMBER 1 may include a display of BASEBALL OR OTHER SPORTS TEAM while NUMBER 2 would include a display of RIVAL OR ALL OTHERS. An example may be YANKEES for Number 1 while this would be associated with a rival such as RED SOX, METS, or DODGERS for the Number 2. Instead of listing a specific team for Number 2, “everyone else”, “our competitors”, “the other guys”, “the other teams” or “the rest of the world” could be displayed. Under this category, NFL teams could be displayed for Number 1 such as GIANTS, VIKINGS or STEELERS. Rival teams could be selected for Number 2.

NUMBER 1 may include a display of YOUR MATE while NUMBER 2 would include a display of ALL OTHERS. An example may be HILLARY for Number 1 while this would be associated with an old mate such as MARY for the Number 2. Instead of listing a specific name for Number 2, “everyone else”, “the other guys”, “the other spouses”, “all others” or “the rest of the world” could be displayed. Under this category, GEORGE could be displayed for Number 1 while EVERYONE ELSE'S MATE could be displayed for Number 2.

NUMBER 1 may include a display of YOUR PRODUCT while NUMBER 2 would include a display of YOUR COMPETITORS OR ALL OTHERS. An example may be GOOGLE for Number 1 while this would be associated with a competitor such as YAHOO for the Number 2. Instead of listing a specific name for Number 2, “everyone else”, “the other guys”, “all competitors”, “all others” or “the rest of the world” could be displayed. Under this category, WAL-MART could be displayed for Number 1 while TARGET could be displayed for Number 2. Another example could be from food products, such as CAMPBELL'S for Number 1 and PROGRESSO for Number 2.

NUMBER 1 may include a display of YOUR HOMETOWN, STATE OR COUNTRY while NUMBER 2 would include a display of YOUR RIVAL OR ALL OTHERS. An example may be PHILADELPHIA for Number 1 while this would be associated with a city such as NEW YORK for the Number 2. Instead of listing a specific name for Number 2, “everyone else”, “the other guys”, “the other cities”, “the other hometowns”, “all others” or “the rest of the world” could be displayed. Under this category, NEW JERSEY could be displayed for Number 1 while CONNECTICUT could be displayed for Number 2. Another example would be UNITED STATES for Number 1 associated with AFGHANISTAN for Number 2.

NUMBER 1 may include a display of YOUR SCHOOL while NUMBER 2 would include a display of YOUR RIVAL OR ALL OTHERS. An example may be HARVARD for Number 1 while this would be associated with YALE for the Number 2. Instead of listing a specific name for Number 2, “everyplace else”, “the other guys”, “the other schools”, “all others” or “the rest of the world” could be displayed. Under this category, WEST POINT could be displayed for Number 1 while ANNAPOLIS could be displayed for Number 2. Another example would be MEDICAL SCHOOL for Number 1 associated with LAW SCHOOLS for Number 2.

NUMBER 1 may include a display of YOUR COMPANY while NUMBER 2 would include a display of YOUR RIVAL OR ALL OTHERS. An example may be GATEWAY for Number 1 while this would be associated with a PC manufacturer such as DELL for the Number 2. Instead of listing a specific name for Number 2, “everyone else”, “the other guys”, “all other competitors”, “the other companies”, “all others” or “the rest of the world” could be displayed. Under this category, GENERAL MOTORS could be displayed for Number 1 while FOREIGN CARS could be displayed for Number 2. Another example would be CANON for Number 1 associated with EPSON for Number 2.

NUMBER 1 may include a display of FRATERNITY OR SORORITY while NUMBER 2 would include a display of YOUR RIVAL OR ALL OTHERS. An example may be ALPHA PHI for Number 1 while this would be associated with a sorority such as CHI OMEGA for the Number 2. Instead of listing a specific name for Number 2, “everyone else”, “the other guys”, “the other fraternities”, “the other sororities”, “the other fraternities and sororities”, “all others” or “the rest of the world” could be displayed.

NUMBER 1 may include a display of YOUR BRANCH, YOUR UNIT OR CLUB while NUMBER 2 would include a display of YOUR RIVAL OR ALL OTHERS. An example may be BOY SCOUT TROOP 142 while this would be associated with a rival troop such as BOY SCOUT TROOP 168 for the Number 2. Instead of listing a specific name for Number 2, “everyone else”, “the other guys”, “the other branches”, “the other troops”, “all others” or “the rest of the world” could be displayed. Under this category, 130^{th} REGIMEN could be displayed for Number 1 while 164^{th} REGIMEN.

NUMBER 1 may include a display of INSTITUTION, HOSPITAL OR SERVICE ORGANIZATION while NUMBER 2 would include a display of YOUR RIVAL OR ALL OTHERS. An example may be MASSACHUSETTS GENERAL HOSPITAL for Number 1 while this would be associated with a hospital such as COLUMBIA GENERAL for the Number 2. Instead of listing a specific name for Number 2, “everyone else”, “the other guys”, “the other hospitals”, “the other medical centers”, “all others” or “the rest of the world” could be displayed. Under this category, GATES FOUNDATION could be displayed for Number 1 while “ALL OTHERS” could be displayed for Number 2.

To summarize, the present invention thus provides diapers and bottom wear. There is a graphical illustration of number one and number two, which are associated with a display of a favored competitor and a disfavored competitor, respectively. The number one and favored competitor is located on the front of the diapers and bottom wear, while the number two and disfavored competitor is locator on the rear of the diapers and bottom wear.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims. For example, organizations include profit and non-profit organizations, including political parties, groups and candidate committees.

What is claimed is:

1. A methodology for presenting competitor favoritism, which comprises:
(a.) presenting a diaper having a front and a rear, said diaper graphically illustrating number one in any known language, numeral system or symbol, on said front of said diaper and number two on said rear of said diaper;
(b.) displaying a favored competitor from two or more competitors on said front of said diaper in proximity to said number one graphic illustration; and,
(c.) displaying at least one disfavored competitor from said two or more competitors on said rear of said diaper in proximity to said number two.

2. The methodology for presenting competitor favoritism of claim 1 wherein said favored competitor is displayed to include a person's name.

3. The methodology for presenting competitor favoritism of claim 1 wherein said favored competitor is displayed to include a name representing an entity selected from the group consisting of a geographic location, a business entity, an institution, and a service organization.

4. The methodology for presenting competitor favoritism of claim 1 wherein said favored competitor is a sports team.

5. The methodology for presenting competitor favoritism of claim 1 wherein said at least one disfavored competitor is displayed as a generic catchall.

6. The methodology for presenting competitor favoritism of claim 5 wherein said generic catchall is selected from the group consisting of “everyone else”, “our competitors”, “the other guy”, “the other team” and “the rest of the world”.

7. The methodology for presenting competitor favoritism of claim 1 wherein said number one is graphically illustrated in a format selected from the group consisting of “NUMBER ONE”, “NUMBER 1”, “No. 1”, “# 1” and “No. One”.

8. The methodology for presenting competitor favoritism of claim 1 wherein said graphically illustrated presentations are printed graphically illustrated presentations and said displays are printed displays.

9. The methodology for presenting competitor favoritism of claim 1 wherein said graphically illustrated presentations are sewn graphically illustrated presentations and said displays are sewn displays.

10. The methodology for presenting competitor favoritism of claim 1 wherein said graphically illustrated presentations are patch graphically illustrated presentations and said displays are patch displays.

11. A methodology for presenting competitor favoritism, which comprises:
(a.) presenting bottom wear selected from the group consisting of underwear, shorts and pants having a front and a rear, said bottom wear graphically illustrating number one in any known language, numeral system or symbol, on said front of said bottom wear and number two on said rear of said bottom wear;
(b.) displaying a favored competitor from two or more competitors on said front of said bottom wear in proximity to said number one graphic illustration; and,
(c.) displaying at least one disfavored competitor from said two or more competitors on said rear of said bottom wear in proximity to said number two.

12. The methodology for presenting competitor favoritism of claim 11 wherein said favored competitor is displayed to include a person's name.

13. The methodology for presenting competitor favoritism of claim 11 wherein said favored competitor is displayed to include a name representing an entity selected from the group consisting of a geographic location, a business entity, an institution, and a service organization.

14. The methodology for presenting competitor favoritism of claim 11 wherein said favored competitor is a sports team.

15. The methodology for presenting competitor favoritism of claim 11 wherein said at least one disfavored competitor is displayed as a generic catchall.

16. The methodology for presenting competitor favoritism of claim 15 wherein said generic catchall is selected from the group consisting of “everyone else”, “our competitors”, “the other guy”, “the other team” and “the rest of the world”.

17. The methodology for presenting competitor favoritism of claim 11 wherein said number one is graphically illustrated in a format selected from the group consisting of “NUMBER ONE”, “NUMBER 1”, “No. 1”, “# 1” and “No. One”.

18. The methodology for presenting competitor favoritism of claim 11 wherein said graphically illustrated presentations are printed graphically illustrated presentations and said displays are printed displays.

19. The methodology for presenting competitor favoritism of claim 11 wherein said graphically illustrated presentations are sewn graphically illustrated presentations and said displays are sewn displays.

20. The methodology for presenting competitor favoritism of claim 11 wherein said graphically illustrated presentations are patch graphically illustrated presentations and said displays are patch displays.
